(19) United States
(12) Patent Application Publication
(10) Pub. No.: US 2025/0295480 A1
(43) Pub. Date: Sep. 25, 2025

(54) METHOD FOR MONITORING CHANGES IN BITE

(71) Applicant: 3SHAPE A/S, Copenhagen K (DK)

(72) Inventors: Peter Dahl Ejby JENSEN, Copenhagen K (DK); Henrik AANÆS, Copenhagen K (DK); Sebastian KULD, Copenhagen K (DK)

(73) Assignee: 3SHAPE A/S, Copenhagen K (DK)

(21) Appl. No.: 18/863,234

(22) PCT Filed: Dec. 12, 2022
(86) PCT No.: PCT/EP2022/085338

§ 371 Date: Nov. 5, 2024

(30) Foreign Application Priority Data
May 6, 2022 (EP) 22171967.7

(51) Int. Cl. A61C 9/00; A61B 6/00; A61B 6/03; A61B 6/51

(52) U.S. Cl. CPC A61C 9/0053; A61B 6/032; A61B 6/512; A61B 6/5217

(57) ABSTRACT

Disclosed is a method for monitoring changes in jaw motion over time. A primary relative jaw motion data set and a secondary relative jaw motion data set are obtained using an intraoral scanner. The method further comprises a computer implemented method, where the computer implemented method comprises the steps of, receiving the primary relative jaw motion data set and the secondary relative jaw motion data set; obtaining a model class representing desired and/or regularising properties of articulation; obtaining a primary model parameters by fitting the primary relative jaw motion data set to the model class; obtaining a secondary model parameters by fitting the secondary relative jaw motion data set to the model class; determining monitoring information based on comparing the primary model parameters with the secondary model parameters, and displaying the monitoring information.

### BACKGROUND

The masticatory system is an incredibly complex musculoskeletal system, comprised of two bony structures, the mandible and the skull, which are connected via the two temporomandibular joints (TMJ). The masticatory system is used in everyday tasks like speaking and chewing, which is vital for the extraction of nutrients from food.

The mechanics of operating the human jaws for chewing food, speaking, etc. is a complex operation involving many individual muscles and two interconnected but individual TMJ connecting the lower jaw (mandible) to the temporal bone on each side of the skull.

The jaw muscles move the jaw in a complex three-dimensional manner during jaw movements. There are three jaw-closing muscles (masseter muscle, temporalis muscle, and medial pterygoid muscle) and four jaw-opening muscles (lateral pterygoid muscle, digastric muscle, mylohyoid muscle and geniohyoid muscle). The basic functional unit of a muscle is the motor unit. The internal architecture of the jaw muscles is complex, with many exhibiting a complex pennate (feather-like) internal architecture. Within each of the jaw muscles, the central nervous system (CNS) appears capable of activating separate compartments with specific directions of muscle fibers. This means that each jaw muscle is capable of generating a range of force vectors (magnitude and direction) required for a particular jaw movement. In the generation of any desired movement, the CNS activates motor units in different muscles. Movements are classified into voluntary, reflex, and rhythmical. Many parts of the CNS participate in the generation of jaw movements. The face motor cortex is the final output pathway from the cerebral cortex for the generation of voluntary movements, such as opening, closing, protrusive, and lateral jaw movements. Reflexes demonstrate pathways that aid in the refinement of a movement and can be used by the higher motor centers for the generation of more complex movements. Mastication or chewing is a rhythmical movement that is controlled by a central pattern generator in the brainstem. The central pattern generator can be modified by sensory information from the food bolus and by voluntary commands from higher centers.

Masticatory movements are complex, consisting of jaw, face, and tongue movements that are driven by jaw, face, and tongue muscles. Changes to the occlusion appear capable of having significant effects on the activity of the jaw muscles and the movement of the jaw joint.

The temporomandibular joint (TMJ) is a movable joint of the mandibular condyle and the glenoid fossa in the skull, with the articular disk interposed between. Movements occur by a combination of rotation (between condyle and disk) and translation (between the condyle-disk complex and the fossa).

This high degree of mobility, in particular of the translatory movement, is reflected in the way in which the disk is attached to the condyle and in the absence of hyaline cartilage. The condyles and fossae are covered by fibrocartilage, and the disk consists of a dense collagen fiber network oriented in different directions related to functional load. Condylar movements are complex and during chewing both condyles are loaded. The TMJ is able to adapt to load changes by remodeling.

Disturbances in the masticatory system may arise from even the smallest deviation in the performance of the different components described above. One of such disturbances may be Temporomandibular disorders (TMDs) which include a variety of different disorders involving the TMJ, the masticatory muscles, and associated structures, separately or together. More than 100 different diseases can affect the musculoskeletal system, and many of these may also involve the TMJ. Some of these are rare, but a few are relatively common, such as osteoarthrosis and osteoarthritis (OA) and rheumatoid arthritis (RA). It is evident that several disorders affecting the TMJ may result in occlusal disturbances. The word “occlusion” represents both static and dynamic tooth relationships and the integrated action of the jaw muscles and temporomandibular joints (TMJ).

Osteoarthrosis (OA) is a degeneration of the TMJ but is, in general, a benign disorder with minor or no symptoms resulting in poor prognosis possibilities. In OA, an inflammatory component is added to the joint degeneration. Acute inflammatory phases associated with pain and dysfunction are usually reversible with simple treatment if diagnosed early.

TMDs may lead to a discomfort for the patient such as pain and decreased functionality of the masticatory system or other areas of the body. Like many other conditions, an early identification and diagnose may lead to a reversible situation for the patient by initiating early treatment.

Early diagnosis of jaw motion related conditions like TMDs are difficult to perform at one specific point in time as there is no clear relationship between one condition and a simple measurement which can be performed to classify TMDs either as arthrogenic or myogenic. This is because patients with a primary joint disorder usually have secondary muscle dysfunction, and patients with a primary muscle disorder may exhibit joint symptoms.

A detailed assessment of the static and dynamic relationships of teeth is important in clinical assessment for all aspects of dental practice. The information will provide an understanding of the specific tooth relationships associated with function and parafunction, upon which treatments may be based. The current method for the clinical occlusion assessment comprises measuring tooth contacts by means of a physical paper at different relative positions of the jaws. Additionally, inspection of clinical signs such as wear on teeth and restorations, muscle pain using a visual analogue scale (VAS) may be made, as well as observations of sounds originating from the TMJ. This is a cumbersome and time-consuming procedure, requiring a lot of manual work and subjective assessment skills from the clinician.

With the emergence of digital solutions in dentistry such occlusal assessments may be performed faster and more accurate using 3D acquisition equipment and computer software.

Accordingly, there exists a need for assessing occlusal changes faster and more accurate.

### SUMMARY

Thus, in one aspect, disclosed herein is a method for monitoring changes in jaw motion over time, wherein the method comprises the steps of,
obtaining a primary relative jaw motion data set at a primary point in time using an intraoral scanner, where the primary relative jaw motion data set represents a relative motion between an upper jaw and a lower jaw.
obtaining a secondary relative jaw motion data set at a secondary point in time using an intraoral scanner, where the secondary relative jaw motion data set represents a relative motion between the upper and the lower jaw,
   wherein the method further comprises a computer implemented method, where the computer implemented method comprises the steps of,
receiving, the primary relative jaw motion data set and the secondary relative jaw motion data set,
obtaining a model class representing desired and/or regularising properties of articulation,
obtaining a primary model parameters by fitting the primary relative jaw motion data set to the model class,
obtaining a secondary model parameters by fitting the secondary relative jaw motion data set to the model class,
determining monitoring information based on comparing the primary model parameters with the secondary model parameters,
displaying the monitoring information.

The computer implemented method disclosed enables a detailed quantification and tracking of dynamic jaw movement over time. This can lead to early identification of any emerging divergence in jaw mobility which may be a warning sign of a potential developing condition. It enables a dentist to take preventive actions at an early stage where most conditions are reversable.

Thus, as will be disclosed and discussed further, the method disclosed enables early identification of occlusal disturbances by quantifying and monitoring mobility of the masticatory system specific jaw movement of a patient over time. By obtaining 3D information of a patient's dentition such as a 3D digital model of the upper and lower jaw it is possible to quantify mandibular movements. By obtaining a plurality of patient specific bite configurations, describing relative jaw motion between the upper and lower jaw, and fitting those bite configurations on a mathematical model approximating the mechanics of masticatory system, a set of patient specific parameters characterizing the masticatory systems of the patient at the time of the data acquisition can be provided. The mathematical model may be a relatively simple model with a limited number of parameters or a more advanced model approaching the natural anatomy of the human masticatory system to a high degree. By acquiring a set of bite configurations at one point in time and obtaining a first set of jaw model parameters it is possible to compare the patient specific jaw movements to a second set of jaw model parameters obtained from a second set of bite configurations obtained at a second point in time. This enables a detailed assessment of patient specific model parameters or border movements of the mandibular.

As used herein the terms ‘primary’ and ‘secondary’ are used to differentiate temporally between features and items. Accordingly, primary relative jaw motion data sets or primary bite scans were all taken at the same substantial point in time, typically at a first patient visit. Secondary relative jaw motion data sets or secondary bite scans are thus a group of bite scans taken at another point in time, e.g. a subsequent patient visit. However, the primary and the secondary relative jaw motion data sets may also be taken at the same patient visit where a treatment has been performed between the obtaining the relative jaw motion data sets. The treatment may be a surgical procedure of mounting a crown or an implant may affect the jaw motion. Although not discussed in further detail tertiary, quaternary, quinary etc. bite scans may be obtained at distinct different points in time.

The primary bite scans may comprise a first primary bite scan and a second primary bite scan. The first primary bite scan and the second primary bite scan cannot have been taken at the exact identical time since the patient needs to move between bite positions. However, for the purpose of e.g. treatments, evaluations and clinical observations, the first primary bite scan and the second primary bite scan can be considered as being taken at the same point in time in order to derive coherent movement data useful for the monitoring purpose as discussed herein.

As discussed herein the relative jaw motion data set contains data describing the relative motion between the upper and lower jaw of the patient. Herein the relative jaw motion data set may also be referred to as jaw motion data set or jaw motion data. The jaw motion data set may be obtained by using an intraoral scanner, e.g. by continuously scanning the upper and the lower jaw while the patient is moving the jaws relative to each other.

In one embodiment the relative jaw motion data set may be obtained by obtaining bite scans. A bite scan is a single discrete scan of the static occlusion of the upper and lower jaw of a patient. Accordingly, multiple (e.g. first, second, third, fourth, etc.) bite scans may represent frames, which in a sequence may be provided as a video stream, e.g. if a dynamic occlusion is obtained by recording it using an intraoral scanner.

Mathematical models simplifying anatomical complexity may be used to map the recorded data. Even if those models do not replicate all aspects of anatomically correct jaw movement, they may still be very useful for in diagnosis and treatment planning of patients as they provide a methodology for tracking and monitoring patient specific changes, such as in the static and/or dynamic occlusion.

By “mathematical modelling” or “modeling”, we understand that we derive/estimate/fit a model, which describes a data set, according to some optimality criteria. The mathematical model may comprise a model class and a fitted model. More specified, the model class is a mathematical expression (intended to model some data) with one or more free parameters to be determined/computed/fitted. The fitted model is a model class with the free parameters determined.

One of the simplest examples is that of a line, where a model class would be y=a*x+b, where x and y are input and output data respectively, and a and b are the free parameters. A fitted model could be y=2*x+3, where a=2 and b=3 are the free parameters.

The model class can have a deterministic and/or a stochastic component, implying that some (or all) part of the model, or model class, can be formulated in terms of a statistical distribution. The latter is the stochastic part. One example could be y=a*x+b+e where e is an element in a normal distribution with mean zero and standard deviation of 1.2. Then a*x+b would be the deterministic part and e the stochastic part. Note that this model can also be described as a normal distribution with a mean of a*x+b, and a standard deviation of 1.2.

“Desired properties of the dynamic occlusion” may be understood as a property we would like to know, such as a contact point distribution of a bite, or the workings of the TMJ.

Term “regularizing property” is a commonly understood term within statistical model fitting where a bias is typically added to an estimate to reduce noise. This is a so-called bias variance trade off. This bias is often based on a priori assumptions or knowledge, and will in a Bayesian statistical frame work be termed “a prior”. A common example of the regularizing property is smoothing data, e.g. a mesh surface, to reduce noise.

In another aspect there is disclosed a method for assessing jaw motion, wherein the method comprises the steps of,
obtaining a primary relative jaw motion data set at a primary point in time using an intraoral scanner, where the primary relative jaw motion data set represents a relative motion between the upper jaw and the lower jaw,
   wherein the method further comprises a computer implemented method, where the computer implemented method comprises the steps of,
receiving the primary relative jaw motion data set,
obtaining a model class representing desired and/or regularising properties of articulation,
obtaining a primary model parameters by fitting the primary relative jaw motion data set to the model class,
determining monitoring information based on comparing the primary model parameters with a reference data set,
displaying the monitoring information.

This provides a solution where a patient's bite may be diagnostically assessed by looking at the motion at one point in time.

For example, jaw motion may be described with a hysteresis, i.e., the relative movement between the upper and the lower jaw is different when opening the jaws compared to when closing the jaws. Absence of the hysteresis may be a sign of a TMJ disease.

Accordingly, in the second aspect the reference data set may in one embodiment comprise a hysteresis criterion. The step of determining monitoring information based on comparing the primary model parameters with the hysteresis criterion may comprise determining whether the primary model parameters describe a hysteresis.

In one embodiment, the reference data set may comprise a Cone Beam computed tomography (CBCT) scan of the upper and/or the lower jaw. By comparing the primary model parameters with the reference data set in form of the CBCT scan, it may be possible to verify accuracy of the primary model parameters. A difference between the primary model parameters and the CBCT scan may be determined, wherein the CBCT scan provides anatomically accurate data of a patient's skull in relation to the lower jaw. Orientation of the TMJ in one configuration, at the time of CBCT scanning, may be directly extracted and compared to the primary model parameters.

In a further embodiment, the reference data set may comprise a treatment plan. The treatment plan may be a simulated movement pattern of the jaw based on an orthodontic treatment. By comparing the primary model parameters with the reference data set in form of the treatment plan, it is possible to determine a difference between the treatment plan and the jaw motion. The determined difference may indicate that the treatment plan needs to be modified. Based on the determined difference the user may create a modified treatment plan based on the primary model parameters.

In yet another embodiment, the reference data set may comprise typical jaw reference parameters describing a motion of the jaw. The typical jaw reference parameters may be obtained, via a statistical method, from a plurality of recorded jaw movements. One example of the statistical method may be line of best fit method. The statistical method may consider one or more of age, gender, ethnicity, living conditions, genes, to achieve a realistic representation of the patient.

The motion of the jaw described by the typical jaw reference parameters may be referred to as an ideal movement trajectory of the jaw. This ideal movement trajectory may describe a smooth motion comprised of only a rotation or only a translation. By comparing the primary model parameters with the ideal movement trajectory of the jaw, both the rotation and the translation may be detected in the monitoring information, indicating a potential TMJ disorder.

Monitoring changes in jaw motion over time may present benefits in tracking progress of an orthodontic treatment. More particularly, teeth movement can be monitored for deviations to a desired movement specified by a planned orthodontic treatment.

According to an embodiment, disclosed herein is a computer implemented method for monitoring changes in jaw motion over time, wherein the method comprises the steps of,
receiving, the primary relative jaw motion data set and the secondary relative jaw motion data set,
determining a change in jaw motion by comparing the secondary relative jaw motion data set to the primary relative jaw motion data set, wherein the secondary relative jaw motion data set corresponds to one or more teeth, wherein the one or more teeth are repositioned compared to position of the one or more teeth corresponding to the primary relative jaw motion data set, and
displaying the determined change in jaw motion.

The repositioned one or more teeth may correspond to a stage of the planned orthodontic treatment, for example the repositioned one or more teeth may correspond to a mid-treatment stage of the planned orthodontic treatment.

The primary relative jaw motion data set may correspond to a primary jaw motion scanned at the first patient visit. The secondary relative jaw motion data set may correspond to a secondary jaw motion scanned at the subsequent patient visit.

The one or more teeth may include at least one tooth that establishes occlusal contact during the primary jaw motion and the secondary jaw motion. Alternatively, the one or more teeth may include at least one tooth that establishes occlusal contact during only the primary jaw motion or only during the secondary jaw motion.

In an embodiment of the disclosure, a non-transitory computer-readable medium is disclosed, comprising instructions which, when executed by a computer, may cause the computer to carry out the method according to any of the presented embodiments.

In an embodiment of the disclosure, a computer program product embodied in the non-transitory computer readable medium is disclosed, the computer program product comprising instructions which, when executed by a computer, may cause the computer to carry out the method according to any of the presented embodiments.

In another aspect there is disclosed a scanner system for intraoral scanning of a dental object. The scanner may comprise a scanning probe for receiving images of the dental object, a peripheral output device for visualising a digital 3D representation of the dental object and a computer processor coupled to the scanning probe and the peripheral output device. The computer processor may receive data from the scanning probe and may output computed data to the peripheral output device. The scanner system may be used by applying the steps of the method as discussed herein.

As may be understood the current disclosure focuses on using an intraoral scanner for obtaining the relative jaw motion data sets in a method for monitoring changes in jaw motion. In another embodiment other means than the intraoral scanner may be used.

Such other means may for example be laboratory-based desktop scanners such as the E-series scanners from 3Shape A/S or x-ray scanning such as CBCT scanners.

### BRIEF DESCRIPTION OF THE DRAWINGS

Aspects of the disclosure may be best understood from the following detailed description taken in conjunction with the accompanying figures. The figures are schematic and simplified for clarity, and they just show details to improve the understanding of the claims, while other details are left out. Throughout, the same reference numerals are used for identical or corresponding parts. The individual features of each aspect may each be combined with any or all features of the other aspects. These and other aspects, features and/or technical effect will be apparent from and elucidated with reference to the illustrations described hereinafter in which:

FIG. 1 shows a schematic view of an embodiment of a method for monitoring changes in jaw motion over time as disclosed herein,

FIGS. 2a-2c shows a 1^{st} example of an embodiment of a method for monitoring changes in jaw motion over time using six degrees of freedom model as a model class,

FIGS. 3a-3c shows a 2^{nd} example of an embodiment of a method for monitoring changes in jaw motion over time using a digital representation of an articulator as a model class,

FIG. 4 shows a 3^{rd} example of an embodiment of a method for monitoring changes in jaw motion over time using a comprehensive rigid body model as a model class,

FIG. 5 shows an embodiment of a border envelope diagram,

FIG. 6 shows and embodiment of another aspect of assessing jaw motion as disclosed herein,

FIG. 7 shows a scanner system for use with a method as disclosed herein.

### DETAILED DESCRIPTION

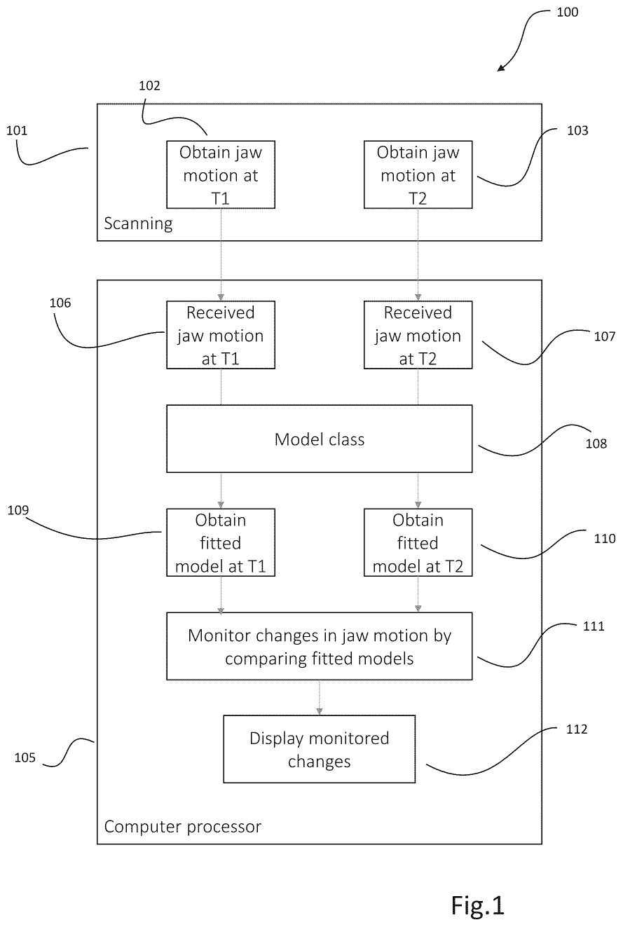

A schematic overview of a monitoring method 100 as disclosed herein is shown in FIG. 1, where the different steps will be discussed further along with additional or alternative embodiment.

In a scanning phase 101 a primary relative jaw motion data set at a certain time T1 is obtained 102 and a secondary relative jaw motion data set at a certain time T2 is obtained 103. The relative jaw motion data represents a relative motion between the upper and the lower jaw of a patient. There is not a predetermined interval in which the two jaw motions should be obtained. In some cases they are obtained with intervals of six months or more, to monitor slow changes in jaw motion over time. The jaw motion data sets may be obtained within days or hours to observe possible changes in jaw motion that may have occurred due to a dental treatment.

The relative jaw motion data sets may be obtained in different ways. In one embodiment, they are recorded directly as the patient is moving the lower jaw relative to the upper jaw. This can for example be obtained by recording a sequence of bite configurations or bite scans. The respective 3D representations of bite configurations can be used to align the 3D representation of the patient's upper jaw and the 3D representation of the patient's lower jaw.

In one embodiment, method may further comprise obtaining at least a first digital 3D representation of at least a part of the upper and a part of the lower jaw of the patient.

The obtained digital 3D representation data may be used during the step of obtaining model parameters by fitting the primary and/or secondary relative jaw motion data to the model class. This may be advantageous, as the obtained digital 3D representation may be used as a reference framework during the fitting procedure of the jaw motion data. The 3D digital representation may improve the mapping of the jaw motion data to the model class as correspondences between jaw motion data and the digital 3D representation may easily be established by an alignment process. Additionally, a signal to noise ratio may be reduced when the 3D digital representation is used, opposed to comparing the relative jaw motion data without the reference framework. This is because the 3D digital representation is a very accurate representation as it is composed of dense data.

In an embodiment, the CBCT scan of the upper and/or lower jaw may be used as the reference framework during the fitting procedure of the jaw motion data. The CBCT scan may improve the mapping of the jaw motion data to the model class as correspondences between jaw motion data and the CBCT scan may easily be established by an alignment process. Additionally, a signal to noise ratio may be reduced when the CBCT scan is used, opposed to comparing the relative jaw motion data without the reference framework.

The digital 3D representation may be obtained prior to obtaining the jaw motion data, by using the intraoral scanner to scan and reconstruct the digital 3D representation of the patient dentition. The digital 3D representation may constitute a full representation of the patients upper and lower jaw or only a portion of the upper and the lower jaw, such as a quadrant.

The digital 3D representation may additionally be obtained by importing the digital 3D representation of at least a part of the patient dentition into the software generated a priori and/or by other means such as the intraoral scanner used to obtain the relative jaw motion data sets. Other examples of generating at least a part of a digital 3D representation of a patient dentition could be scanning a physical impression or a gypsum model, CBCT (x-rays) or any other suitable way of generating a digital 3D representation.

In one embodiment, additional digital 3D representations may be obtained in relation to obtaining jaw motion data at the different points in time. This may be particularly advantageous, if the patient has/is undergoing orthodontic treatment resulting in movement of the teeth or other factors having resulted in major changes to patient dentition in between the different points in time.

In case that no major changes of the patient's dentition are expected, the same 3D digital representation may be utilized by the computer implemented method for both set of jaw motion data as correspondences between the primary and secondary jaw motion data and an identical 3D digital representation can be established.

In one embodiment, the method for obtaining model parameters by fitting the primary and/or secondary relative jaw motion data to the model class may be performed without obtaining the 3D digital representation of at least a part of the patient's upper and lower jaw. This may be done by directly acquiring jaw motion data as a consecutive series of small 3D data patches, which may be the bite scans as discussed herein, containing partial information of the dentition in the upper jaw and partial information of the patient dentition in the lower jaw.

Accordingly, in one embodiment, an initial data patch, such as the first primary bite scan, may be used as a primary reference framework for the model class while correspondences between the initial data patch and subsequent data patches, e.g.

second, third, fourth etc. primary bite scans, may be established either directly or indirectly through registration of intermediate scan patches. This process in also known as simultaneous localization and mapping. Similarly, this may also be applied to the secondary bite scan in order to establish a secondary reference framework.

Accordingly, by determining framework correspondences which are both in the primary and the secondary reference framework, a common coordinate system can be established, which subsequently allows for the primary model parameters and the secondary model parameters to be compared in order to determine the monitoring information. The correspondences may for example be landmarks, data points or other features.

The reference framework established by the embodiment above mimics the step of obtaining a digital 3D representation while acquiring jaw motion data simultaneously. It may be advantageous to apply additional data processing steps to the individual data patches to assist and improve the registration process to account for the relative movement between the jaws. Such data processing could be a real time identification of upper and lower jaw in each scan patch such that sub part of the same scan patch may be registered to different sub parts of prior scan patches. This may have the advantage of omitting the separate step of obtaining the digital 3D representation.

Accordingly, in one embodiment, the step(s) of obtaining the primary and/or secondary relative jaw motion data set may comprise obtaining at least a first and a second primary and/or secondary bite scan at a primary and/or secondary point in time respectively, each comprising at least a part of the upper and the lower jaw in relation to each other at different jaw motion positions using an intraoral scanner.

Subsequently in one embodiment the step of obtaining the primary and/or secondary relative jaw motion data set may further comprise a first primary and/or secondary alignment of the upper and lower jaw of the digital 3D representation based on the first primary and/or secondary bite scan, and a second primary and/or secondary alignment of the upper and lower jaw of the digital 3D representation based on the second primary and/or secondary bite scan.

The alignment process may comprise an initial alignment and then be followed by optimization, for example by using an Iterative Closest Point (ICP) algorithm. For more information see for example “Method for registration of 3-D shapes” by Paul J. Besl and Neil D. Mckay, Proc. SPIE 1611, Sensor Fusion IV: Control Paradigms and Data Structures, 586 (Apr. 30, 1992).

This method is particularly advantageous because it allows for aligning the 3D digital representation of the patients upper and lower jaw to the bite configuration data patch, wherein the bite configuration data patch may be a 3D data patch only comprising partial information of the patient's dentition in the upper and the lower jaw.

More than two different bite configurations may be required in order to determine or estimate the occlusal contact movement of the patient's jaws relative to each other, such as three, four, five, six, seven, eight, nine, ten etc.

The bite configurations can be recorded using an intraoral 3D scanner, such as 3Shape's TRIOS scanner. The dentist may ask the patient to bite the upper jaw and lower jaw together. As the patient briefly holds the bite for the first configuration, the dentist will perform a scan of the patient's teeth by means of the intraoral scanner to acquire the first 3D representation. After releasing the first bite, the dentist may ask the patient for a second bite. While the patient bites together for a second configuration, the dentist will perform a scan of the patient's teeth by means of the intraoral scanner to acquire a second 3D representation. More bite configurations may be desired or required for providing enough data to properly fit the mathematical model and obtain patient specific model parameters, the dentist may ask the patient to bite the teeth together a third, fourth, fifth etc. time and perform a scan for each bite.

In one embodiment, it might be particularly of advantage to record jaw motion data containing at least some information that includes the relative position of the upper and lower jaw in their extreme or maximal range positions for a full range of jaw motion analysis. Such maximal range positions may be one or more of the following:
centric relation (CR)
intercuspal position (ICP)
retruded contact position (RCP)
protruded jaw position (RCP)
postural jaw position (P)
maximum jaw opening (O)
hinge arc of opening (H).

The patient may be instructed and/or assisted to guide the jaw to a certain maximal range position. The jaw motion data may be recorded during the movement from one position to another and/or at the different positions. Some extreme positions might be difficult to record using an intraoral scanner, hence a subsequent step of applying a model class to fit the jaw motion data may be of advantage, as some positions may be derived from the fitted mathematical description.

Subsequently to obtaining the relative jaw motion data set, the method further comprises a computer implemented method for executing a number of steps. The computer implemented method may be executed on a computer processor 105, or on a plurality of computer processor.

After scanning 101 the primary and secondary relative jaw motion data sets are sent to a computer and received 106, 107 in e.g. a software application running on the computer processor.

The acquisition of multiple bite configurations may comprise a consecutive sequence of at least two 3D representations of bite configurations of the patient's jaws in respective occlusions.

By taking such a consecutive sequence of bite configurations, it is possible to obtain a high number of interdependent bite configurations which may provide a suitable data collection for fitting the mathematical model precisely.

The primary and secondary relative jaw motion data set could be compared directly. However, the recordings of the relative jaw motion data, such as the bite configurations, may be noisy, which is the case with most physical measurements of geometry. The noise may partially originate from sensor noise, semi-transparent material e.g., saliva or teeth when they are scanned.

As a consequence, the signal to noise ratio in the section of the observed jaw motion may be of lower quality. The relative jaw motion, or patient specific motion, may be obtained by scanning the molar region on one side of the mouth. Here the signal to noise ratio of a rotation around an axis pointing away from the face typically has less observability.

The obtained jaw motion data may not have recordings of all possible/likely relative jaw motions/positions of the jaws relative to each other as this would require the patient performing all possible movements while being scanned. Therefore, an interpolation of relative jaw motion data may take place from the multiple bite configurations. Performing such interpolations between obtained bite configurations typically provides linear interpolation of data, which is often inaccurate since jaw motion is rarely linear. Thus, providing a mathematical model, e.g. a model class of possible/likely motions, given the observed data will reduce the risk of errors when interpolating.

Additionally, extrapolation of data may be performed using model parameters and the model class to predict bite configurations that are outside of the jaw motion data set. The accuracy of extrapolated data may be improved by selecting a suitable model class, potentially including limitations or ranges assigned to the various model class parameters. The range of some model class parameter may additionally be interdependent. Examples of such limitations or ranges could be obtained from statistical values known in the field, such as:

Approximate range of jaw movement in adults:
RCP to ICP 0.5-2.0 mm
ICP to O 40-70 mm
RCP to H 15-20 mm
P to ICP 2-4 mm
ICP to P 5-10 mm.

For more sophisticated model classes, known physical factors such as maximum muscle or tendon tension may provide differently specified intervals for the model class parameters.

Fitting a mathematical model consisting of possible/likely relative motions, to the obtained bite configuration data may be performed digitally. Accordingly, a model class 108 representing desired and/or regularising properties of articulation is obtained.

By fitting each of the primary relative jaw motion and the secondary relative jaw motion to the model class, the primary model parameters 109 and secondary model parameters 110 may be obtained.

The mathematical model, e.g. the model class, may describe the relative motion of the jaw. It may express a Euclidean transformation, i.e., a rotation and translation in 3D space. It is proposed to constrain the space of possible rotations and translations by either disallowing some configurations or making some configurations less likely than others. The latter applies if a probabilistic approach to describing relative jaw motion is taken. These constrains, in the space of Euclidian transformations, are captured by the mathematical models selected to fit to the data. At any instant in time during the jaw movements, the jaw can be described as rotating around an instantaneous (i.e. at the given point in time) axis of rotation but typically six-degrees-of-freedom models will accurately describe the complexity of such movement.

The mathematical model may be fitted by a non-linear iterative minimization scheme, e.g., a version of the damped newton methods. Other numerical schemes are also viable. The objective function, used for such a fitting is an error measure between the measured data and how that data would be, if it were constrained to a given mathematical model. An example of such a measure is a 2-norm (or a robust version hereof) on a point-to-point basis between the captured scan points and their projection onto the mathematical model.

Accordingly, a solution to make a mathematical model constraining the possible or likely Euclidean relative positions over time is disclosed. This has the advantage that noise, not consistent with this model, can efficiently be filtered away or at least considerably reduced. Additionally, the interpolation between measured relative jaw positions, i.e., motions, is facilitated. This is also the case when extrapolating to the probable motions in space.

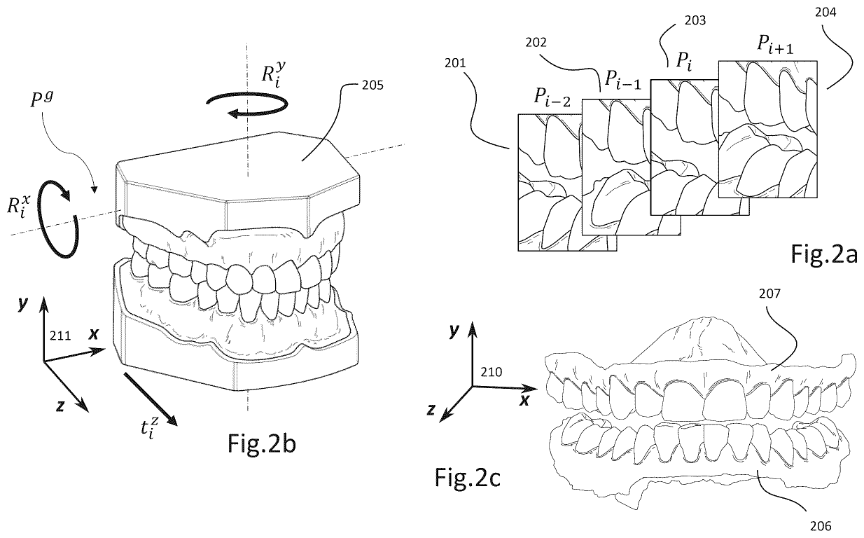

Accordingly, different types of model classes may be used as disclosed herein. For example, the model class may describe six degrees of freedom, three rotational degrees and three translational degrees. The first example described with respect to FIGS. 2a-2c is one such embodiment.

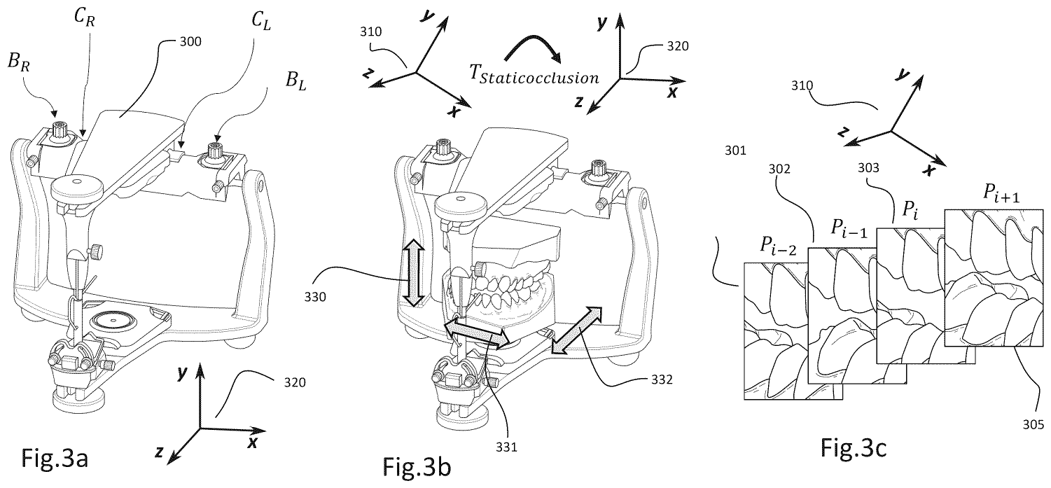

Such model class may in another embodiment be a digital representation of an articulator, where the second example described herein with respect to FIGS. 3b-3c is an embodiment thereof.

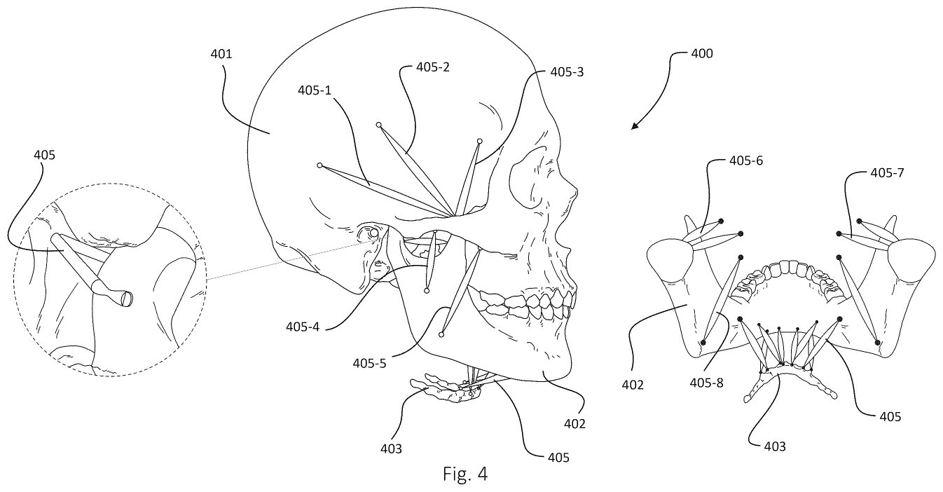

In yet another embodiment, the model class may be a digital representation of a standardized human jaw. This is for example described in the third example herein together with FIGS. 4.

One embodiment of using a mathematical model for fitting bite configurations to a model is described as a first example with respect to FIGS. 2a, 2b and 2c. This embodiment may for example be used in the method described herein with respect FIG. 1.

The input to our estimation, is a plurality of bite configurations 201, 202, 203 and 204 as a sequence of poses, Pᵢ, of the lower jaw 206, in a coordinate system 210 where the upper jaw 207 is fixed (upper jaw coordinate system). Here a pose has 6 degrees of freedom, and consist of a rotation, Rᵢ and a translation, tᵢ, where i denotes the ‘time’ or ‘sequence number’. That is

Pᵢ=[Rᵢtᵢ]

A model 205 may be fitted to these poses, consisting of a global pose, i.e., not dependent on time, P^{g}. This global pose is thought of a positioning of the TMJ's in relation to the upper jaw and may define a model specific coordinate system 211. Then for each Pᵢ, a 3 degree of freedom pose may be fitted, consisting of a rotation around the x-axis, Rᵢ^{x}, a rotation around the y-axis Rᵢ^{y} and a translation in the direction for the z axis, tᵢ^{z}. Then denoting the translation P^{g} as

P^{g}=[R^{g}t^{g}]

The Rᵢ and tᵢ are modelled as follows:

$$\begin{matrix}{R}_{i} = {( {R}^{g} )}^{T} {R}_{i}^{x} {R}_{i}^{y} {R}^{g} \\ {t}_{i} = {( {R}^{q} )}^{T} {R}_{i}^{χ} {R}_{i}^{y} ( {t}^{q} + {t}_{i}^{Z} ) - {t}^{q}\end{matrix}$$

This modelling is equivalent to first applying the ‘local’ time varying transformation, and then the global transformation to the points on the lower jaw. Noting that the combined pose at time i is given by Pᵢ=[Rᵢtᵢ]. This is seen to be a 3 degree of freedom for each of the poses (plus 6 degrees for the whole system), thus almost constraint half the degrees of freedom of the system. This model is fitted to each of the Pᵢ in a least squares fashion.

An interpretation of the above model is as follows; it is noted that the poses at time i, Pᵢ, are given in an ‘arbitrary’ frame of reference, which is here assumed to be the coordinate system chosen for the upper jaw. P^{g} is the transformation from this upper jaw coordinate system to the model coordinate system. The system in which the axis of rotation (associated with Rᵢ^{x} and Rᵢ^{y}i) and translation tᵢ^{z} are naturally represented. Rᵢ^{x} is the rotation around the x-axis in the model coordinate system. Therefore, when estimating P^{g}, we are de facto also estimating this model's coordinate system and thus finding the x-axis of rotation that best describes all the data. Similar may be done for the y-axis and Rᵢ^{y}, and the z-axis and tᵢ^{z}. This implies that Rᵢ^{x} is a rotation axis that may be used for all time instances i, an axis defined as the x-axis in the model coordinate system. Given this x-axis, Rᵢ^{x} is a one-parameter function of an angle, θᵢ^{x}. This can be described as a quaternion;

$$[ cos \frac{{θ}_{i}^{x}}{2} , 0 , sin \frac{{θ}_{i}^{x}}{2} , 0 , 0 ]$$

similarly, for Rᵢ^{y} and angle θᵢ^{y} :

$$[ cos \frac{{θ}_{i}^{y}}{2} , 0 , sin \frac{{θ}_{i}^{y}}{2} , 0 ]$$

And tᵢ^{z}, with a distance δᵢ:

[0,0,δᵢ]

In summary, P^{g} is the estimation of where the 2 axis of rotation and the direction of translation should be relative to the upper jaw system, with the constraint that they should be perpendicular.

Given P^{g}, which is constant for all time instances, the time dependent vector may be expressed:

[θᵢ^{x}, θᵢ^{y},δᵢ]

In a second example, an embodiment of a mathematical model is disclosed representing a physical dental articulator may be used as illustrated in FIGS. 3a, 3b and 3c. A dental articulator has screws, hinges and scales to adjust the articulator's movement range and therewith setup the specific jaw motion possibilities in the articulator as well as intrinsic parameters of the model construction restricting a free movement. In the case of the popular Artex CR articulator 300 from Amann Girrbach AG, there are at least four specific parameters of interest: the Bennett angle Left (BL), Bennett angle Right (BR), and the horizontal Condylar Path Left and Right, (CL and CR) as displayed in FIG. 3a. These parameters, BL, BR, CL, CR, may be referred to as the static model parameters as they are setup once for the entire articulation and are independent of time. In other dental articulator types, the available parameters and their adjustment possibilities may differ, but if a mathematical model can be made to represent an articulator, then the parameters can be fitted using the methods described here.

To find the parameters that are the closest match to the plurality of bite configurations, data fitting methods can be used to minimize the difference between the motion provided by the articulator model and the motion provided by the obtained bite configurations. In order to do that in practice, a transformation can be found that positions the 3D digital representation of the patient's dentition in their static occlusion coordinate system 310 in the articulator's coordinate system 320 FIG. 3b.

The transformation can be defined as Tstaticocclusion and is the transformation from Tpose to Tarticulator. An initial guess for this transformation can be found in several ways, including using teeth segmentation to identify the teeth in the scan and matching their location to a template model placed in the articulator. Alternatively, an estimated occlusal plane from the 3D digital dental models and their center of mass provides another approach.

The transformation Tstaticocclusion is included into the data fitting optimization to make sure that fitted articulator parameters are based on an optimal placement of the scan in the articulator. As it is a ridged transformation it contains six (6) parameters, three (3) for translation and three (3) for rotation. Like the four (4) articulator parameters these are static parameters that are fitted once for the entire articulation.

Besides the static parameters as outlined above, there are also dynamic parameters that describe the position of the lower jaw with regards to the upper jaw at a given point in time i. In the case of the ACR articulator there are four parameters: the Occlusal Opening 330, Laterotrusion 331 and the Protrusion 332 and Retrusion 332. These parameters correspond to the location the incisal pin is adjusted to when doing the articulator motion by means of the articulator model, yielding the pose of the upper jaw 3D digital representation in relation to the static pose of the lower jaw 3D digital representation. This corresponds directly to the data obtained in the plurality of bite configurations 301, 302, 303, 304, showing relative transformation between the lower jaw and the upper jaw in pose Pᵢ shown in FIG. 3c. As we need to fit the articulator parameters from the obtained poses in the plurality of bite configurations, we also need to take the dynamic parameters into account and add them to the data fitting operation.

Once the data fitting problem is outlined, a possible implementation to solve it may be sketched as:
setup the articulator model to its default position describing a standard setting,
find an initial guess for Tstaticocclusion, e.g. by using teeth segmentation or occlusal plane estimate.

For each step in the optimization it may be possible to iterate on the static articulator parameters and Tstaticocclusion parameters.

For each image in the motion scan it may be possible to:
iterate on the dynamic articulator parameters,
calculate the error as a difference between the pose found in the i′th bite configuration and the corresponding pose given by the articulator model function using the current dynamic parameters. Calculation can e.g. be a difference between the eight corners of the scans bounding box transformed with both pose transformations.

The optimization can be done when the parameters have converged or a maximum number of steps has been taken.

In the third example, shown in FIG. 4, a comprehensive rigid body model 400 encompassing the whole masticatory region combined with a detailed representation of the TMJ may be used to approximate the human anatomy to a high degree. This model contains a high number of parameters and is based on the work of Sagl et al. (doi: 10.3389/fphys.2019.01156). Here, the bone structures, such as the skull 401, hyoid bone 403 and the lower jaw (mandible) 402, were modelled as rigid bodies. Inertial properties of the mandible were estimated from mesh geometry with an assumed mandibular mass of 200 g (Langenbach and Hannam, 1999). The hyoid bone 403 and skull 401 were kept static for all presented simulations and therefore no inertia properties had to be defined. Muscles were represented as Hill-type point-to-point muscles 405 (Hill, 1953; Peck et al., 2000; Hannam et al., 2008). For clarity, only a few of the Hill-type point-to-point muscles are shown with a reference sign, but it will be understood that the FIG. 4 shows many more. Since these muscle models apply forces in the one-dimensional direction of the force vectors defined by an origin and insertion point, larger muscles were split up into multiple strings to more accurately mimic activation of muscle compartments. The included muscles may be posterior 405-1, medial 405-2 and anterior part 405-3 of the temporal muscle; superior head of the lateral pterygoid muscle 405-6 and inferior head of the lateral pterygoid muscle 405-7, superficial 405-5 and deep 405-4 masseter muscles, as well as the medial pterygoid muscle 405-8. Other muscles that may also be included (although not shown) may be, but not limited to, anterior digastric muscle, geniohyoid muscle, anterior and posterior mylohyoid muscle.

In addition to the examples, such models could also be augmented with statistical probabilities, i.e., what is the probability density function of relative jaw motion. To motivate, many of the problems patient specific motion shall and could address relate to the most likely bites, e.g., for tooth fillings or orthodontic treatments. This does not imply that a patient cannot have a relative jaw movement, which practically never happens, but could in theory, which should be prioritized down e.g., with regards to the daily chewing movements, when doing treatments.

The embodiment presented herein goes beyond the mathematical modelling of mechanical articulators. Where it is noted that, these articulators are themselves mechanical (and not digital) models of the human jaw physiological system. Obviously, extensions include more flexible modelling of the TMJ, which has a considerable amount of flexibility built into it, e.g., in relation to a metallic joint. As an example, the TMJ has elements of hysteresis in it, i.e., the path of the jaw moving forward is not the same as moving backwards. Other factors that could be included, are temporal constraints, e.g., in the form of a Kalman filter of the lower jaw poses. Also, the lower jaw is not perfectly rigid, even though it is often modelled as such (e.g. a scan or a gypsum cast), and can deform slightly, e.g., if one bites hard. This could also well be included in the models fitted.

Identification of chances in the mobility of the patient's jaw mobility may be quantified by performing an acquisition of a first set of bite configurations at a first point in time T1 and a second set of bite configurations at a second point in time T2. For each set of bite configurations it may be possible to perform the procedure of fitting the same mathematical model to both sets of bite configurations.

Using the obtained fitted model at T1 109, e.g. the primary model parameters and the obtained fitted model at T2 110, e.g. the secondary model parameters, it is possible to determine monitoring information such as changes in jaw motion by comparing the two fitted models 111.

The monitoring information may then be displayed to the user 112 to indicate relevant changes in jaw motion. The monitoring information may be displayed in different ways, e.g. it may simply display the model parameters and/or indicate changes therein. Such changes may be indicated or highlighted where they are above a certain criterion.

Alternatively or additionally, the monitoring information may be visualised by showing a color/heat map indicating where critical changes occur or some discrepancies in the jaw motion, while playing a 3D digital motion video of the upper and lower jaw.

By using the model described in example 1, several forms of data analysis may be performed. Statistics on the time dependent vector [θᵢ^{x}, θᵢ^{y}, δᵢ] may give indications of the patient's jaw ‘agility’. As an example, the min-max range of the θᵢ^{x}; θᵢ^{y} could be directly translatable to the patients range of motion.

This can also be used in a “change detection setting”. That is if a patient's health status is monitored over time, e.g., two jaw motion data scans six months apart. Then comparing the statistics of [θᵢ^{x}, θᵢ^{y}, δᵢ] obtained at a primary time T1 and [θᵢ^{x}, θᵢ^{y}, δᵢ] obtained at secondary time T2. This analysis may reveal any changes in the mobility of the jaws between the two points in time. The identification of small perturbations mobility of the jaw may be early sings of potential issues in the patient's masticatory system.

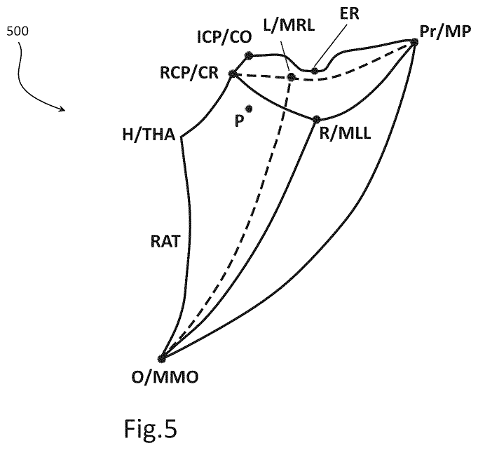

One comparison of such jaw dynamics could be done according to a border movement analysis. Mandibular movements are anatomically limited by the temporomandibular joints (TMJ), the surrounding and involved ligaments, the neuromuscular system, and the teeth. The boundaries or extremes of the mandibular movements may be referred to as “border movements” or “envelope of movements”. All possible mandibular movements occur within its boundaries. One way to visualize and analyse the border movements of the mandible is by means of a Posselt diagram. Via the documentation of various but distinct positions of the mandible from the different views, a Posselt diagram can create a three-dimensional representation of the different position the mandible, including the maximal movements the mandible is capable of. The full range of jaw movement may be described in three planes by tracing the path of the lower incisal point as the jaw is guided through the border paths. The border path traces the maximum range of jaw movement, which is determined by the jaw muscles, ligaments, limitations of the TMJ and the teeth. The teeth define the top section of the border diagram, which is of particular interest in restorative dentistry, as the relationship between ICP (IP) and CO (RCP) is diagrammatically indicated in FIG. 5.

The border diagram may be displayed in sagittal, frontal, and horizontal planes. The sagittal plane view of the lower jaw's border movement in dentate individuals is captured on the lower incisor teeth and provides features of particular interest.

This type of border diagram may be constructed based on the mathematical model used as a representation of the plurality of bite configurations. If the acquired bite configurations cover at least some of the mandibular extreme movements, a border diagram may be obtained based on the sequence of bite configurations acquired at time T1 and another boarder diagram could be obtained based on the bite configurations acquired at time T2. The two border diagrams may be compared by displaying a contour map of the differences on a display. The contour map may be obtained by aligning the two diagram and calculating the distances. The contour map may be displayed as a color overlay on one of the boarder diagrams.

The use of a mathematical representation framework for describing the mandibular movements relative to the skull enables a broad range of analysis options. One option is to map the direct trajectory of mandibular along the movement from one position to another, for example between two border movements. This would allow for detailed examination of the model parameters along that particular path of the lower jaw. Changes in the patient's masticatory system may be indicated if the trajectory of the jaw following a particular movement is diverging from an expected path or if changes in a particular movement path can be observed over time.

Accordingly, in one embodiment, the step of comparing the primary model parameter and the secondary model parameter comprises using border movement. Such border movement may for example be described by a border movement diagram, such as a Posselt's diagram.

One embodiment of a border movement diagram 500 is shown in FIG. 5. The border movement describes a movement volume defined by the boundaries of the patient's mandibular movements. The movement volume is an expression of the natural relative jaw movements that can be performed by the patient.

FIG. 5 shows Posselt's envelope of motion of a patient. Other types of visualizations could also be used. Certain border points are marked, such as the centric relation CR, the centric occlusion CO, the maximum right lateral position MRL, the maximum left lateral position MLL, the maximum protrusion MP, the edge-to-edge relationship ER and the maximum mouth opening MMP. In addition, movement segments such as the true hinge axis THA and the rotation after translation RAT are indicated. The basic mandibular movements are rotation, translation and lateral movements and can be documented using the Posselt's envelope of motion. The mandible can open up to 20 mm while the condyles remain in their terminal hinge position only rotating around that hinge axis THA. Further opening causes the condyles to leave their glenoid fossa and translate down the articular eminence. This movement is indicated by the RAT.

In another embodiment of lateral movements, the condyle on the working side rotates around a sagittal axis with minimal lateral shift. The condyle on the non-working moves noticeable to the medial side (Bennett movement) as the condyle descends forward and downwards along the medial wall of the glenoid fossa. This side shift can happen at different stages of the downward movement e.g. before the downward movement (immediate side shift=ISS), in the beginning of the downward movement of the condyle (early side shift=ESS), or gradually along the entire downward movement (progressive/distributed side shift=PSS). The form of the Bennett movement has an influence on occlusal surface and is to be considered to prevent the interference of the cusps during lateral excursion. However, each person is only able to conduct one of those forms as that movement is dictated by the anatomy of the masticatory system. Using the border movement can be done in different manners and can also be combined.

In one embodiment, a model class is established representing the border movement. E.g. the six degrees of freedom class model described in the first example may be established with the limitations defined by border movement. Thus, when the relative jaw motions are applied, the class model can also be used for extrapolating border movement that may not have been obtained by the recorded relative jaw motion.

Alternatively or additionally, the primary relative jaw motion data set and the secondary relative jaw motion data set, at least partly, represent one or more border movement positions of the upper and lower jaw. The border movement positions may thus be obtained by for example scanning the relative position of the jaws when they are in one of the points described in FIG. 5, i.e. the centric relation CR, the centric occlusion CO, the maximum right lateral position MRL, the maximum left lateral position MLL, the maximum protrusion MP, the edge-to-edge relationship ER and the maximum mouth opening MMP. This can for example be obtained as a bite scan.

As may be understood, the primary model parameters and the secondary model parameters may thus in one embodiment describe/represent border movements and border movement positions, and these may thus be compared in order to obtain monitoring information.

Using the border envelope 500 even further monitoring comparisons may be done. In one embodiment, where the primary model parameters represent a primary border envelope and the secondary model parameters represent a secondary border envelope, a heat map comparison may be done to visually show differences in movement above a certain threshold in places where considerable changes have occurred. Also in some embodiments, respective parts of the primary and secondary border envelope are compared, this can for example be cross sections thereof, border movement during occlusion or border movement while opening and closing the jaws.

Another analysis approach to performing change detections in mandibular movements could be analyzing differences in axis of rotation. This approach would not be based on estimating the boundaries or extremes of mandibular movements. By using the model described in the first example and aligning the coordinate systems of the upper jaw at the two sessions (based on a stable reference such as landmarks, individual segmented teeth or alike), then it may be possible to compare the P^{g} for the two sessions. This is beneficial as any change in the TMJ would be reflected in the comparison. Alternatively, using the P^{g} from the first session in the last would directly enable comparison of motion, i.e. the [θᵢ^{x}, θᵢ^{y}, δᵢ], between the two scanning sessions.

By applying the mathematical representation of the articulator as described in the second example, changes or differences in the mandibular movements could be monitored by tracking and comparing the static model parameters obtained at a first and a second point in time, which in case of the example using the Artex CR articulator from Amann Girrbach AG, may be the left Bennett angle BL and right Bennett angle BR as well as the Left and Right angle of the condylar path, CL and CR.

Usually, analysis of mandibular movements within the normal ranges of operation e.g., the positions typically used in everyday function of the jaw, are of great interest to quantify and monitor. One approach to obtain a sequence of bite configurations could be to record the relative position of the patients jaws contentiously using an intraoral scanner while the patient is chewing a gum or suitable rubber/silicone piece. This will allow an acquisition of a plurality of bite configurations during the natural chewing motion of a patient.

Another way of analysing changes in the jaw movement pattern of the patient over time would be to use more sophisticated mathematical models approximating the human anatomy as described in the third example. Here, model parameters are based on representing the physical structure of the masticatory system, and changes or direct parameters extracted from such a model might contain information about a condition of a particular muscle, ligament etc.

Complementary to the plurality of bite configurations obtained using an intraoral scanner, additional data might be included in the model optimization to increase the accuracy of the mathematical model used. Such additional data might be recordings of extraoral face scans, which could provide additional information as teeth in the face scans can be aligned with the obtained 3D digital representations. X-ray imaging of the patient may provide information about the TMJ in one static position, which could be added to the model parameter optimization with a high weight or confidentiality, such that this data point might be taken strongly into account. Bite force measurements is another additional data source which may provide supporting information of teeth contact points, which additionally may be added to the optimization procedure. Other types of data recordings providing information of the relative position of the patients upper and lower jaw may strengthening the modelling.

Machine learning and neural networks may be used to recognize specific changes in jaw movement patterns and providing a probability score of a specific clinical condition. This requires significant amount of clinical training data where patients have been monitored before, during and after a condition associated with the masticatory system has been clinically diagnosed.

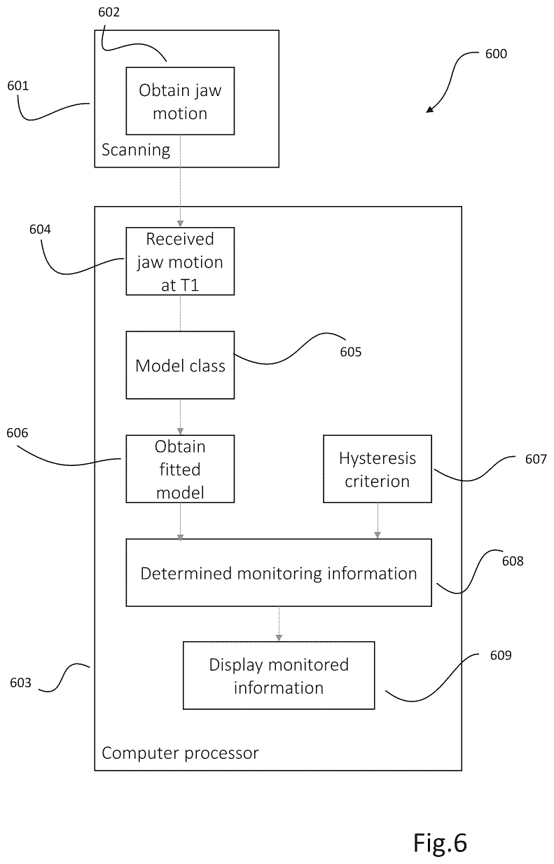

In another aspect, as shown in the FIG. 6, a method for assessing jaw motion 600 is disclosed. In a scanning step 601 a primary relative jaw motion data set 602 at a primary point in time is obtained using an intraoral scanner. The primary jaw motion data set represents a relative motion between the upper jaw and the lower jaw.

The primary relative jaw motion data set is sent 604 to a computer processor 603 for further processing in a computer implemented method.

A model class representing desired and/or regularising properties of articulation 605 may be provided in the processor, e.g. stored for use, or imported into the processor when needed. Primary model parameters can be obtained 606 by fitting the primary relative jaw motion data set to the model class.

A reference data set, defining a hysteresis criterion 607 may be provided and monitoring information can be determined 608 based on comparing the primary model parameters with the reference data set.

The monitoring information can then be subsequently displayed to the user 609, which can be done as disclosed previously. As discussed previously the monitoring information may be displayed in many different ways, e.g. it may simply display the model parameters and/or indicate changes therein. Such changes may be indicated or highlighted if they are above a certain criterion, such as the hysteresis criterion.

Alternatively or additionally, the monitoring information may be visualised by showing a color/heat map indicating where critical changes occur or some discrepancies are present in the jaw motion while playing a 3D digital motion video of the upper and lower jaw.

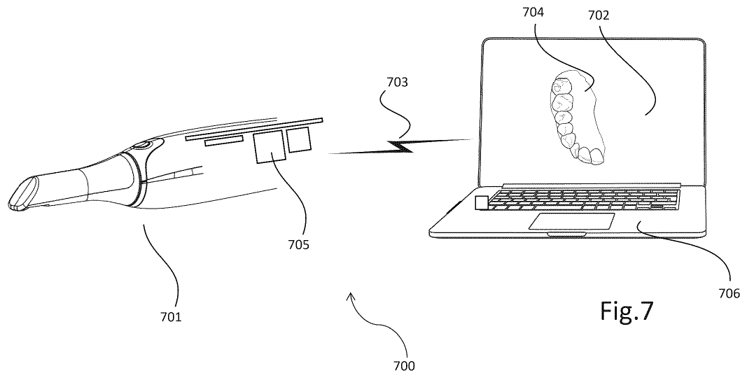

An embodiment of a scanner system 700 for intraoral scanning of the dental object is shown in FIG. 7. The scanner system 700 may be used for performing the method for monitoring changes in jaw motion over time and the method for assessing jaw motion as disclosed herein.

The scanner system may comprise a scanning probe 701 for receiving images of the dental object.

A laptop computer 706, having a peripheral output device 702 in the shape of a monitor for visualising the digital 3D representation 704 of the dental object can be provided in wireless communication with the scanning probe. Alternative a desktop computer or other computing device, e.g. a tablet, can be provided in communication with the scanning probe. The communication may be done wirelessly as described or wired.

Instead of displaying the digital 3D representation 704 the display may display the monitoring information as disclosed here, for example as an overlay on the digital 3D representation 704.

A computer processor 705 and other electronic hardware may be arranged in the scanning probe and enable the wireless communication via a controller 706 in the scanning probe and a controller 707 in the laptop computer 706.

The laptop may receive data from the scanning probe and output computed data to the monitor.

The computer processor provided in the scanning probe and in the laptop computer is typically made up of different types of electronic hardware. The electronic hardware may include microprocessors, microcontrollers, digital signal processors (DSPs), field programmable gate arrays (FPGAs), programmable logic devices (PLDs), gated logic, discrete hardware circuits, and other suitable hardware configured to perform the various functionality described throughout this disclosure. Computer program shall be construed broadly to mean instructions, instruction sets, code, code segments, program code, programs, subprograms, software modules, applications, software applications, software packages, routines, subroutines, objects, executables, threads of execution, procedures, functions, etc., whether referred to as software, firmware, middleware, microcode, hardware description language, or otherwise.

The scanner system disclosed herein may be an intraoral scanning device such as the TRIOS series scanners from 3Shape A/S. The scanning device may employ a scanning principle suitable for intraoral scanning such as triangulation-based scanning, confocal scanning, focus scanning, ultrasound scanning, stereo vision, structure from motion, or any other scanning principle. In an embodiment, the scanning device is operated by projecting a pattern and translating a focus plane along an optical axis of the scanning device and capturing a plurality of 2D images at different focus plane positions such that each series of captured 2D images corresponding to each focus plane forms a stack of 2D images. The acquired 2D images are also referred to herein as raw 2D images, wherein raw in this context means that the images have not been subject to image processing. The focus plane position is preferably shifted along the optical axis of the scanning system, such that 2D images captured at a number of focus plane positions along the optical axis form said stack of 2D images (also referred to herein as a sub-scan) for a given view of the object, i.e. for a given arrangement of the scanning system relative to the object. After moving the scanning device relative to the object or imaging the object at a different view, a new stack of 2D images for that view may be captured. The focus plane position may be varied by means of at least one focus element, e.g., a moving focus lens. The scanning device is generally moved and angled during a scanning session, such that at least some sets of sub-scans overlap at least partially, in order to enable stitching during scanning. The result of stitching is the digital 3D representation of a surface larger than that which can be captured by a single sub-scan, i.e. which is larger than the field of view of the 3D scanning device. Stitching, also known as registration, works by identifying overlapping regions of 3D surface in previous sub-scans/recorded 3D surface and transforming the new sub-scans to a common coordinate system such that the overlapping regions match, finally yielding the digital 3D model. An Iterative Closest Point (ICP) algorithm may be used for this purpose. Another example of a scanning device is a triangulation scanner, where a time varying pattern is projected onto the dental object and a sequence of images of the different pattern configurations are acquired by one or more cameras located at an angle relative to the projector unit.

The scanning device comprises one or more light projectors configured to generate an illumination pattern to be projected on a three-dimensional dental object during a scanning session. The light projector(s) preferably comprises a light source, a mask having a spatial pattern, and one or more lenses such as collimation lenses or projection lenses. The light source may be configured to generate light of a single wavelength or a combination of wavelengths (mono-or polychromatic). The combination of wavelengths may be produced by using a light source configured to produce light (such as white light) comprising different wavelengths. Alternatively, the light projector(s) may comprise multiple light sources such as LEDs individually producing light of different wavelengths (such as red, green, and blue) that may be combined to form light comprising the different wavelengths. Thus, the light produced by the light source may be defined by a wavelength defining a specific color, or a range of different wavelengths defining a combination of colors such as white light. In an embodiment, the scanning device comprises a light source configured for exciting fluorescent material of the teeth to obtain fluorescence data from the dental object. Such a light source may be configured to produce a narrow range of wavelengths. In another embodiment, the light from the light source is infrared (IR) light, which is capable of penetrating dental tissue. The light projector(s) may be DLP projectors using a micro mirror array for generating a time varying pattern, or a diffractive optical element (DOF), or back-lit mask projectors, wherein the light source is placed behind a mask having a spatial pattern, whereby the light projected on the surface of the dental object is patterned. The back-lit mask projector may comprise a collimation lens for collimating the light from the light source, said collimation lens being placed between the light source and the mask. The mask may have a checkerboard pattern, such that the generated illumination pattern is a checkerboard pattern. Alternatively, the mask may feature other patterns such as lines or dots.

The scanning device preferably further comprises optical components for directing the light from the light source to the surface of the dental object. The specific arrangement of the optical components depends on whether the scanning device is a focus scanning apparatus, a scanning device using triangulation, or any other type of scanning device. A focus scanning apparatus is further described in EP 2 442 720 B1 by the same applicant, which is incorporated herein in its entirety.

The light reflected from the dental object in response to the illumination of the dental object is directed, using optical components of the scanning device, towards the image sensor(s). The image sensor(s) are configured to generate a plurality of images based on the incoming light received from the illuminated dental object. The image sensor may be a high-speed image sensor such as an image sensor configured for acquiring images with exposures of less than 1/1000 second or frame rates in excess of 250 frames pr. second (fps). As an example, the image sensor may be a rolling shutter (CCD) or global shutter sensor (CMOS). The image sensor(s) may be a monochrome sensor including a color filter array such as a Bayer filter and/or additional filters that may be configured to substantially remove one or more color components from the reflected light and retain only the other non-removed components prior to conversion of the reflected light into an electrical signal. For example, such additional filters may be used to remove a certain part of a white light spectrum, such as a blue component, and retain only red and green components from a signal generated in response to exciting fluorescent material of the teeth.

The dental scanning system preferably further comprises a processor configured to generate scan data (intra-oral scan data) by processing the two-dimensional (2D) images acquired by the scanning device. The processor may be part of the scanning device. As an example, the processor may comprise a Field-programmable gate array (FPGA) and/or an Advanced RISC Machines (ARM) processor located on the scanning device. The scan data comprises information relating to the three-dimensional dental object. The scan data may comprise any of: 2D images, 3D point clouds, depth data, texture data, intensity data, color data, and/or combinations thereof. As an example, the scan data may comprise one or more point clouds, wherein each point cloud comprises a set of 3D points describing the three-dimensional dental object. As another example, the scan data may comprise images, each image comprising image data e.g. described by image coordinates and a timestamp (x, y, t), wherein depth information can be inferred from the timestamp. The image sensor(s) of the scanning device may acquire a plurality of raw 2D images of the dental object in response to illuminating said object using the one or more light projectors. The plurality of raw 2D images may also be referred to herein as a stack of 2D images. The 2D images may subsequently be provided as input to the processor, which processes the 2D images to generate scan data. The processing of the 2D images may comprise the step of determining which part of each of the 2D images are in focus in order to deduce/generate depth information from the images. The depth information may be used to generate 3D point clouds comprising a set of 3D points in space, e.g., described by cartesian coordinates (x, y, z). The 3D point clouds may be generated by the processor or by another processing unit. Each 2D/3D point may furthermore comprise a timestamp that indicates when the 2D/3D point was recorded, i.e., from which image in the stack of 2D images the point originates. The timestamp is correlated with the z-coordinate of the 3D points, i.e., the z-coordinate may be inferred from the timestamp. Accordingly, the output of the processor is the scan data, and the scan data may comprise image data and/or depth data, e.g. described by image coordinates and a timestamp (x, y, t) or alternatively described as (x, y, z). The scanning device may be configured to transmit other types of data in addition to the scan data. Examples of data include 3D information, texture information such as infra- red (IR) images, fluorescence images, reflectance color images, x-ray images, and/or combinations thereof.

Although some embodiments have been described and shown in detail, the disclosure is not restricted to such details, but may also be embodied in other ways within the scope of the subject matter defined in the following claims. In particular, it is to be understood that other embodiments may be utilized, and structural and functional modifications may be made without departing from the scope of the present invention.

Benefits, other advantages, and solutions to problems have been described herein with regard to specific embodiments. However, the benefits, advantages, solutions to problems, and any component(s)/unit(s) that may cause any benefit, advantage, or solution to occur or become more pronounced are not to be construed as critical, required, or essential features or components/elements of any or all the claims or the invention. The scope of the invention is accordingly to be limited by nothing other than the appended claims, in which reference to a component/unit/element in the singular is not intended to mean “one and only one” unless explicitly so stated, but rather “one or more.” A claim may refer to any of the preceding claims, and “any” is understood to mean “any one or more” of the preceding claims.

It is intended that the structural features of the devices described above, either in the detailed description and/or in the claims, may be combined with steps of the method, when appropriately substituted by a corresponding process.

As used, the singular forms “a,” “an,” and “the” are intended to include the plural forms as well (i.e. to have the meaning “at least one”), unless expressly stated otherwise. It will be further understood that the terms “includes,” “comprises,” “including,” and/or “comprising,” when used in this specification, specify the presence of stated features, integers, steps, operations, elements, and/or components, but do not preclude the presence or addition of one or more other features, integers, steps, operations, elements, components, and/or groups thereof. It will also be understood that when an element is referred to as being “connected” or “coupled” to another element, it can be directly connected or coupled to the other element but an intervening elements may also be present, unless expressly stated otherwise. Furthermore, “connected” or “coupled” as used herein may include wirelessly connected or coupled. As used herein, the term “and/or” includes any and all combinations of one or more of the associated listed items. The steps of any disclosed method is not limited to the exact order stated herein, unless expressly stated otherwise.

It should be appreciated that reference throughout this specification to “one embodiment” or “an embodiment” or “an aspect” or features included as “may” means that a particular feature, structure or characteristic described in connection with the embodiment is included in at least one embodiment of the disclosure. Furthermore, the particular features, structures or characteristics may be combined as suitable in one or more embodiments of the disclosure. The previous description is provided to enable any person skilled in the art to practice the various aspects described herein. Various modifications to these aspects will be readily apparent to those skilled in the art, and the generic principles defined herein may be applied to other aspects.

The claims are not intended to be limited to the aspects shown herein, but is to be accorded the full scope consistent with the language of the claims, wherein reference to an element in the singular is not intended to mean “one and only one” unless specifically so stated, but rather “one or more.” Unless specifically stated otherwise, the term “some” refers to one or more.

1-30 (canceled)

31. A method for monitoring changes in jaw motion over time, wherein the method comprises the steps of,
obtaining a primary relative jaw motion data set at a primary point in time using an intraoral scanner, where the primary jaw motion data set represents a relative motion between an upper jaw and a lower jaw.
obtaining a secondary relative jaw motion data set at a secondary point in time using an intraoral scanner, where the secondary relative jaw motion data set represents a relative motion between the upper and the lower jaw,
wherein the method further comprises a computer implemented method, where the computer implemented method comprises the steps of,
receiving the primary relative jaw motion data set and the secondary relative jaw motion data set,
obtaining a model class representing desired and/or regularising properties of articulation,
obtaining a primary model parameters by fitting the primary relative jaw motion data set to the model class,
obtaining a secondary model parameters by fitting the secondary relative jaw motion data set to the model class,
determining monitoring information based on comparing the primary model parameters with the secondary model parameters,
displaying the monitoring information.

32. The method according to claim 31 wherein the model class describes six degrees of freedom.

33. The method according to claim 31, wherein the model class is a digital representation of an articulator.

34. The method according to claim 31, wherein the model class is a digital representation of a standardized human jaw.

35. Method according to claim 31, wherein the model class is deterministic.

36. Method according to claim 31, wherein the model class is stochastic.

37. Method according to claim 31, wherein the monitoring information is displayed by numerically displaying at least one of the primary and secondary model parameters.

38. Method according to claim 37, wherein monitoring information comprises changes between the primary model parameters and the secondary model parameter.

39. Method according to claim 31, wherein the monitoring information is visualised as a heat map.

40. Method according to claim 31, wherein comparing the primary model parameter and the secondary model parameter comprises using a border movement.

41. Method according to claim 40, wherein the primary relative jaw motion data set and the secondary relative jaw motion data set at least partly represent one or more border movement positions of the upper and lower jaw.

42. Method according to claim 40, wherein the primary model parameters and the secondary model parameters represent border movements.

43. Method according to claim 31, wherein the step(s) of obtaining the primary and/or secondary relative jaw motion data set comprises obtaining at least a first and a second primary and/or secondary bite scan at a primary and/or secondary point in time respectively, each comprising at least a part of the upper and the lower jaw in relation to each other at different jaw motion positions using an intraoral scanner.

44. Method according to claim 43, wherein a primary reference framework is established by determining correspondences between the at least first and the second primary bite scans and a secondary reference framework is established by determining correspondences between the at least first and the second secondary bite scans.

45. Method according to claim 44, wherein framework correspondences are determined between the primary reference framework and the secondary reference framework.

46. Method according to claim 43, wherein obtaining the primary and/or secondary relative jaw motion set further comprises a first primary and/or secondary alignment of the upper and lower jaw of the at least first digital 3D representation based on the first primary and/or secondary bite scan, and a second primary and/or secondary alignment of the upper and lower jaw of the at least first digital 3D representation based on the second primary and/or secondary bite scan.

47. A computer implemented method for monitoring changes in jaw motion over time, wherein the method comprises the steps of,
receiving by a processor, a primary relative jaw motion data set and a secondary relative jaw motion data set;
determining a change in jaw motion by comparing the secondary relative jaw motion data set to the primary relative jaw motion data set, wherein the secondary relative jaw motion data set corresponds to one or more teeth, wherein the one or more teeth are repositioned compared to position of the one or more teeth corresponding to the primary relative jaw motion data set, and
displaying the determined change in jaw motion.

48. A scanner system for intraoral scanning of a dental object, comprising a scanning probe for receiving images of the dental object, a peripheral output device for visualising a digital 3D representation of the dental object and a computer processor coupled to the scanning probe and the peripheral output device, wherein the computer processor is configured to receive data from the scanning probe and output computed data to the peripheral output device, wherein the scanner system is configured for use in the method according to claim 31.

49. A non-transitory computer-readable medium comprising instructions which, when executed by a computer, cause the computer to carry out the method claim 31.

50. A computer program product embodied in a non-transitory computer readable medium, the computer program product comprising instructions which, when executed by a computer, cause the computer to carry out the method according to claim 31.
